(19) United States
(12) Patent Application Publication
(10) Pub. No.: US 2025/0146033 A1
(43) Pub. Date: May 8, 2025

(54) METHOD FOR SYNTHESIZING DIACYLGLYCEROL

(71) Applicants: SOUTH CHINA UNIVERSITY OF TECHNOLOGY, Guangzhou, Guangdong (CN); YHSN Technology Ltd., Guangzhou, Guangdong (CN)

(72) Inventors: Yonghua WANG, Guangzhou (CN); Xuan LIU, Guangzhou (CN); Bo YANG, Guangzhou (CN); Weifei WANG, Guangzhou (CN); Dongming LAN, Guangzhou (CN); Riming LUO, Guangzhou (CN)

(73) Assignees: SOUTH CHINA UNIVERSITY OF TECHNOLOGY, Guangzhou, Guangdong (CN); YHSN Technology Ltd., Guangzhou, Guangdong (CN)

(21) Appl. No.: 18/835,704

(22) PCT Filed: Oct. 18, 2021
(86) PCT No.: PCT/CN2021/124382

§ 371 Date: Aug. 2, 2024

(30) Foreign Application Priority Data
Nov. 6, 2020 (CN) 202011232198.8

(51) Int. Cl. C12P 7/6454

(52) U.S. Cl. CPC C12P 7/6454

(57) ABSTRACT

A method for synthesizing diacylglycerol, comprising the following steps: (1) mixing an oil, a lipase and a solvent, and carrying out a hydrolysis reaction to obtain a hydrolyzate; and (2) under the action of a diacylglycerol lipase, performing an esterification reaction on the hydrolyzate obtained in step (1) with glycerol; the lipase in step (1) is lipase MAS1 or MAS1-H108A.

### FIELD OF THE INVENTION

The present disclosure relates to the field of chemical synthesis, in particular to a method for synthesizing diacylglycerol.

### BACKGROUND OF THE INVENTION

Obesity has gradually become an extremely serious public health problem in countries around the world. Diacylglycerol (DAG), as a functional oil, can reduce excessive accumulation of body fat and promote lipid metabolism in the human body, thereby attracting widespread attention. The DAG is a structural lipid formed when one of the fatty acids in triacylglycerol (TAG) is replaced with a hydroxyl group. The DAG consists of two structural isomers, 1,2-DAG and 1,3-DAG, and is part of a natural composition of edible oil. The DAG is listed as a safe food (GRAS) by the US Food and Drug Administration (FDA) and is widely applied in the food industry.

Main methods for preparing the DAG include a glycerolysis method, an esterification method, and a hydrolysis method. For the glycerolysis method, the glycerol and the TAG are used as substrates, and a glycerolysis reaction is catalyzed by an enzymatic method, whereby a reaction product mainly including the DAG is obtained. Due to presence of a large amount of the glycerol in a glycerolysis system, the viscosity of the system increases, thereby affecting enzyme catalysis. For the esterification method, the fatty acids and the glycerol are catalyzed to react by the enzymatic method, and products containing DAG are obtained. However, high-purity fatty acids need to be prepared before esterification, whereby compared with other preparation methods, the method is usually more complex in a preparation process, which will increase preparation costs and reaction control difficulty to a certain extent. Moreover, generation of the TAG in an esterification process greatly reduces purity of the DAG. Because the TAG and the DAG are difficult to separate, the content of the TAG affects the purity of the DAG, and it is crucial to control the content of the TAG. For the hydrolysis method, the TAG is used as the substrate, the lipase at positions 1 and 3 are selected for partial hydrolysis, and the DAG is prepared by controlling the degree of hydrolysis. However, the degree of the hydrolysis is difficult to control in actual industrial production, which adds certain difficulty to the hydrolysis method. Therefore, it is difficult to prepare the high-purity DAG by a single preparation method, and multiple technical means are needed for collaborative preparation.

CN 105400837 A discloses a method for preparing DAG through enzymatic catalysis. A method of firstly moderately hydrolyzing an oil and then performing esterification on a hydrolyzate and glycerol is adopted, and the content of the DAG in an obtained product is 60%-65%. However, the preparation technology needs to strictly control the content of the fatty acids in the hydrolysis process to be 26-30 wt %. A controllable range is very narrow, and improper control may lead to excessive hydrolysis of the DAG, producing more fatty acids and reducing the purity of the DAG. Besides, the lipase used in the esterification process of the method is TAG lipase, which will generate the TAG in the esterification process, resulting in a decrease in the content of the DAG and an increase in the content of the TAG. Therefore, it is imperative to seek an economical and convenient high-purity preparation method.

### SUMMARY OF THE INVENTION

Based on this, the objective of the present disclosure is to provide a method for synthesizing high-purity DAG.

A specific technical solution is as follows:
a method for synthesizing DAG includes the following steps:
step (1), mixing an oil, a lipase and a solvent, and performing a hydrolysis reaction to obtain a hydrolyzate; and
step (2), under the action of a partial glyceride lipase, performing an esterification reaction on the hydrolyzate obtained in step (1) with glycerol, where
the lipase in step (1) is TAG lipase MAS1 or MAS1-H108A.

In some preferred embodiments, the lipase is the TAG lipase MAS1-H108A.

In some embodiments, the TAG lipase MAS1 or MAS1-H108A is derived from Streptomycetes sp.Strain W007.

In some embodiments, the content of fatty acids in the hydrolyzate obtained in step (1) is controlled to be greater than 45 wt %, preferably greater than 50 wt %, further preferably greater than 60 wt %. The inventors of the present disclosure have found that by controlling the content of the fatty acids in the hydrolyzate obtained in step (1) to be greater than 60 wt %, the DAG with purity greater than 80% can be prepared.

In some embodiments, the content of the TAG in the hydrolyzate obtained in step (1) is controlled to be less than 22 wt %, preferably less than 20 wt %, further preferably less than 15 wt %.

In some embodiments, the partial glyceride lipase is one or a mixture of more than two of lipase SMG1, lipase PCL, and partial glyceride lipase AOL.

In some embodiments, the lipase SMG1 is from malassezia.

In some embodiments, the lipase PCL is from penicillium.

In some embodiments, the partial glyceride lipase AOL is from aspergillus oryzae.

In some embodiments, the solvent is water or a buffer with pH of 6-8.

In some embodiments, the temperature of the hydrolysis reaction in step (1) is 20-60° C.

In some embodiments, pH of the hydrolysis reaction in step (1) is 6-8.

In some embodiments, the temperature of the esterification reaction in step (2) is 30-50° C.

In some embodiments, pH of the esterification reaction in step (2) is 6-8.

In some embodiments, a mass ratio of the oil to the solvent in step (1) is 1: (0.1-5), and further preferably the mass ratio is 1: (0.5-2).

In some embodiments, the mass ratio of the glycerol to the hydrolyzate in step (2) is (0.5-5): 1, further preferably (0.5-1.5): 1.

In some embodiments, an addition quantity of the lipase in step (1) is 50-1000 U/g of the mass of the oil, further 400-600 U/g.

In some embodiments, an addition quantity of the partial glyceride lipase in step (2) is 50-1000 U/g of the total mass of an esterification reaction mixture, further 100-400 U/g.

In some embodiments, the hydrolyzate is an oil phase obtained by layering a reaction system after the hydrolysis reaction is completed. The oil phase (hydrolyzate) does not require further separation and purification, and is directly subjected to the esterification reaction with the glycerol.

In some embodiments, after the obtained esterification reaction in step (2) is completed, the esterification product is subjected to separation and purification.

In some embodiments, the separation and the purification are separation and purification through molecular distillation.

Compared with the prior art, the method disclosed by the present disclosure has the following beneficial effects:
the inventors of the present disclosure have found that by selecting the TAG lipase MAS1 or MAS1-H108A especially MAS1-H108A for hydrolyzing the oil, which has a strong ability to hydrolyze the TAG, the content of the DAG in the hydrolyzate can be greater than that of the TAG; then, the obtained hydrolyzate is directly subjected to the esterification reaction with the glycerol without separating the TAG from the fatty acids, then the partial glyceride lipase is selected to catalyze the esterification reaction, and finally, high-purity DAG with low content of the TAG can be prepared by the method of the present disclosure.

In addition, the present disclosure adopts the method for catalyzing first hydrolysis and then esterification of the oil to prepare the DAG, without worrying about excessive hydrolysis of the oil, and the method is simple to operate, strong in controllability, low in production costs, and convenient in industrial production.

### DETAILED DESCRIPTION OF THE EMBODIMENTS

Experimental methods without specifying specific conditions in the following examples of the present disclosure were performed in accordance with conventional conditions, or in accordance with conditions recommended by manufacturers. Various commonly used chemical reagents used in the examples are all commercially available products.

All technical and scientific terms used in the present disclosure have the same meanings as those commonly understood by those skilled in the art to which the present disclosure pertains, unless otherwise defined. The terms used in the description of the present disclosure are only for the purpose of describing specific embodiments and are not intended to limit the present disclosure.

The terms “comprise/include” and “have” of the present disclosure, as well as any variations thereof, are intended to cover non-exclusive inclusions. For example, a process, a method, an apparatus, a product, or a device that includes a series of steps is not limited to listed steps or modules, but optionally further includes steps that are not listed, or optionally further includes other steps inherent to the process, the method, the product, or the device.

The term “a plurality of” referred to in the present disclosure refers to two or more. “And/or” describes an association relationship of associated objects, indicating that three types of relationships can exist, for example A and/or B can indicate three situations: existence of A alone, coexistence of A and B, and the existence of B alone. A character “/” generally indicates that the front-rear associated objects are an “or” relationship.

During long-term research on lipase MAS1 and MAS1-H108A, the inventors of the present disclosure unexpectedly discovered for the first time that, unlike conventional lipases, these two lipases (especially MAS1-H108A) have a greater tendency to hydrolyze TAG than DAG. Based on this discovery, the inventors of the present disclosure combined their long-term experience and extensive scientific exploration to ultimately obtain a method for synthesizing DAG of the present disclosure, which includes the following steps:
step (1), mixing oil, lipase and solvent, and performing hydrolysis reaction to obtain a hydrolyzate; and
step (2), under the action of partial glyceride lipase, performing esterification reaction on the hydrolyzate obtained in step (1) with glycerol, where
the lipase in step (1) is lipase MAS1 or MAS1-H108A.

The lipase MAS1 described in the present disclosure is a wild-type lipase derived from actinomycetes Streptomycetes sp.Strain W007, and the lipase MAS1-H108A is a mutant of the wild-type lipase MAS1. The lipase MAS1 or MAS1-H108A used in the method of the present disclosure can be purchased or prepared according to conventional methods in the art.

In some implementations, the content of fatty acids in the hydrolyzate obtained in step (1) is controlled to be greater than 45 wt %, further, the content of the fatty acids can be selectively controlled to be greater than 46 wt %, 47 wt %, 48 wt %, 49 wt %, 50 wt %, 52 wt %, 53 wt %, 54 wt %, 55 wt %, 56 wt %, 57 wt %, 58 wt %, 59 wt %, 60 wt %, 61 wt %, 61 wt %, 63 wt %, 64 wt %, or 65 wt %, and higher-purity DAG can be prepared. The inventors of the present disclosure have found that by controlling the content of the fatty acids in the hydrolyzate obtained in step (1) to be greater than 60 wt %, the DAG with purity greater than 80% can be prepared.

In some implementations, the content of the TAG in the hydrolyzate obtained in step (1) is controlled to be less than 22 wt %, further, the content of the TAG can be selectively controlled to be less than 21 wt %, 20 wt %, 19 wt %, 18 wt %, 17 wt %, 16 wt %, 15 wt %, 14 wt %, 13 wt %, 12 wt %, 11 wt %, 10 wt %, or 9 wt %, and higher-purity DAG can be prepared.

In some implementations, the partial glyceride lipase is one or a mixture of more than two of lipase SMG1, lipase PCL, and partial glyceride lipase AOL.

In some implementations, the solvent is water or a buffer with a pH of 7-8. The buffer with a pH of 7-8 may be a conventional buffer, such as a phosphate buffer with a pH of 7-8.

In some implementations, the temperature of the hydrolysis reaction in step (1) is 20-60° C., and further, the temperature of the hydrolysis reaction is 30-50° C. Specifically, the temperature of the hydrolysis reaction is 20° C., 22° C., 24° C., 26° C., 28° C., 30° C., 32° C., 34° C., 36° C., 38° C., 40° C., 42° C., 44° C., 46° C., 48° C., or 50° C.

In some implementations, the pH of the hydrolysis reaction in step (1) is 6-8.

In some implementations, the temperature of the esterification reaction in step (2) is 30-50° C., and further, the temperature of the esterification reaction is 35-45° C.; and specifically, the temperature of the esterification reaction is 30° C., 32° C., 34° C., 36° C., 38° C., 40° C., 42° C., 44° C., 46° C., 48° C., or 50° C.

In some implementations, the pH of the esterification reaction is 6-8.

In some implementations, a mass ratio of the oil to the solvent in step (1) is 1: (0.1-5), and further preferably the mass ratio is 1: (0.1-2); and specifically, the mass ratio of the oil to the solvent is 1:0.1, 1:0.2, 1:0.3, 1:0.4, 1:0.5, 1:0.6, 1:0.7, 1:0.8, 1:0.9, and 1:1.

In some implementations, a mass ratio of the glycerol to the hydrolyzate in step (2) is (0.5-5): 1, further preferably (0.5-1.5): 1; and specifically, the mass ratio of the glycerol to the hydrolyzate is 1:0.1, 1:0.2, 1:0.3, 1:0.4, 1:0.5, 1:0.6, 1:0.7, 1:0.8, 1:0.9, and 1:1.

In some implementations, an addition quantity of the MAS1 lipase in step (1) is 50-1000 U/g of the mass of the oil, further 100-800 U/g, 200-700 U/g, 300-600 U/g, or 400-600 U/g.

In some implementations, an addition quantity of the partial glyceride lipase in step (2) is 50-1000 U/g of the total mass of an esterification reaction mixture, further 50-800 U/g, 50-600 U/g, 50-400 U/g, 100-300 U/g.

In some implementations, the hydrolyzate is an oil phase obtained by layering a reaction system after the hydrolysis reaction is completed, and layering is performed by a conventional layering method.

After the obtained esterification reaction in step (2) is completed, an esterification product is subjected to separation and purification; and further, the separation and the purification are separation and purification through molecular distillation by a conventional method.

The present disclosure will be further described in detail in combination with specific examples.

The sources or reference preparations of various enzymes used in the following embodiments are as follows:

preparation of TAG lipase MAS1: Research on gene cloning, expression and properties of lipase MAS1 [D]. South China University of Technology, 2016.

Preparation of TAG lipase MAS1-H108A: Li Linlin, Research on preparation of immobilized lipase MAS1-H108A and application thereof in catalytic synthesis of alpha-linolenic acid-rich TAG [D]. South China University of Technology, 2019.

Partial glyceride lipase PCL: partial glyceride lipase PCL and partial glyceride lipase PCL-I260R have been disclosed in patent “CNIO8642026A: Partial glyceride lipase mutant and application thereof”.

Partial glyceride lipase AOL: partial glyceride lipase AOL and partial glyceride lipase AOL-V269D have been disclosed in patent “CNIO8504643A: Partial glyceride lipase derived from aspergillus oryzae and preparation method and crystal structure thereof”.

TAG lipase TL100L: derived from Novozymes.

### Example 1

(1) Hydrolysis: soybean oil and a phosphate buffer solution with a pH of 7.5 were added in a conical flask with a stopper, in a mass ratio of 1:0.5, were placed on a constant temperature magnetic stirrer at a speed of 500 rpm and preheated at 40° C. for 10 min, after preheating was completed, 500 U/g of TAG lipase MAS1-H108A (based on a total mass of oil) was added, after 8 h, sampling was performed for detection, standing for layering was performed, an oil phase was taken as a hydrolyzate, an aqueous phase was recovered, and the obtained oil phase did not need separation of fatty acids and glycerides. The obtained hydrolyzate contained 60.2% of the fatty acids, 20.5% of the DAG, and 12.3% of the TAG.

(2) Esterification: the hydrolyzate and the glycerol were added to the conical flask with a stopper in a mass ratio of (1:1), were placed on the constant temperature magnetic stirrer at a speed of 500 rpm and preheated at 40° C. for 10 min, and after preheating was completed, 200 U/g of partial glyceride lipase PCL was added. The obtained esterification product contained 9.5% of the fatty acids, 55.4% of the DAG, and 12.1% of the TAG. After separation and purification through molecular distillation, the content of the DAG was 82.07%.

### Example 2

(1) Hydrolysis: soybean oil and a phosphate buffer solution with a pH of 7.5 were added in a conical flask with a stopper, in a mass ratio of 1:0.5, were placed on a constant temperature magnetic stirrer at a speed of 500 rpm and preheated at 40° C. for 10 min, after preheating was completed, 500 U/g of TAG lipase MAS1-H108A (based on a total mass of oil) was added, after 12 h, sampling was performed for detection, standing for layering was performed, an oil phase was taken as a hydrolyzate, an aqueous phase was recovered, and the obtained oil phase did not need separation of fatty acids and glycerides. The obtained hydrolyzate contained 70.1% of the fatty acids, 15.6% of the DAG, and 10.5% of the TAG.

(2) Esterification: the hydrolyzate and the glycerol were added to the conical flask with a stopper in a mass ratio of (1:1), were placed on the constant temperature magnetic stirrer at a speed of 500 rpm and preheated at 40° C. for 10 min, and after preheating was completed, 200 U/g of partial glyceride lipase PCL was added. The obtained esterification product contained 13.2% of the fatty acids, 53.0% of the DAG, and 10.0% of the TAG. After separation and purification through molecular distillation, the content of the DAG was 84.13%.

### Example 3

(1) Hydrolysis: soybean oil and a phosphate buffer solution with a pH of 7.5 were added in a conical flask with a stopper, in a mass ratio of 1:0.5, were placed on a constant temperature magnetic stirrer at a speed of 500 rpm and preheated at 40° C. for 10 min, after preheating was completed, 500 U/g of TAG lipase MAS1-H108A (based on a total mass of oil) was added, after 24 h, a hydrolyzate was sampled for detection, standing for layering was performed, an oil phase was taken as the hydrolyzate, an aqueous phase was recovered, and the obtained oil phase did not need separation of fatty acids and glycerides. The obtained hydrolyzate contained 80.8% of the fatty acids, 10.1% of the DAG, and 8.2% of the TAG.

(2) Esterification: the hydrolyzate and the glycerol were added to the conical flask with a stopper in a mass ratio of (1:1), were placed on the constant temperature magnetic stirrer at a speed of 500 rpm and preheated at 40° C. for 10 min, and after preheating was completed, 200 U/g of partial glyceride lipase PCL was added. The obtained esterification product contained 15.1% of the fatty acids, 50.2% of the DAG, and 8.1% of the TAG. After separation and purification through molecular distillation, the content of the DAG was 85.79%.

### Example 4

(1) Hydrolysis: soybean oil and a phosphate buffer solution with a pH of 7.5 were added in a conical flask with a stopper, in a mass ratio of 1:0.5, were placed on a constant temperature magnetic stirrer at a speed of 500 rpm and preheated at 40° C. for 10 min, after preheating was completed, 500 U/g of TAG lipase MAS1 (based on a total mass of oil) was added, after 36 h, sampling was performed for detection, standing for layering was performed, an oil phase was taken as a hydrolyzate, an aqueous phase was recovered, and the obtained oil phase did not need separation of fatty acids and glycerides. The obtained hydrolyzate contained 63% of the fatty acids, 18.7% of the DAG, and 13.3% of the TAG.

(2) Esterification: the hydrolyzate and the glycerol were added to the conical flask with a stopper in a mass ratio of (1:1), were placed on the constant temperature magnetic stirrer at a speed of 500 rpm and preheated at 40° C. for 10 min, and after preheating was completed, 200 U/g of partial glyceride lipase PCL was added. The obtained esterification product contained 12.20% of the fatty acids, 52.3% of the DAG, and 13.1% of the TAG. After separation and purification through molecular distillation, the content of the DAG was 80.02%.

### Example 5

(1) Hydrolysis: soybean oil and a phosphate buffer solution with a pH of 7.5 were added in a conical flask with a stopper, in a mass ratio of 1:0.5, were placed on a constant temperature magnetic stirrer at a speed of 500 rpm and preheated at 40° C. for 10 min, after preheating was completed, 500 U/g of TAG lipase MAS1 (based on a total mass of oil) was added, after 48 h, sampling was performed for detection, standing for layering was performed, an oil phase was taken as a hydrolyzate, an aqueous phase was recovered, and the obtained oil phase did not need separation of fatty acids and glycerides. The obtained hydrolyzate contained 73% of the fatty acids, 13.1% of the DAG, and 11.8% of the TAG.

(2) Esterification: the hydrolyzate and the glycerol were added to the conical flask with a stopper in a mass ratio of (1:1), were placed on the constant temperature magnetic stirrer at a speed of 500 rpm and preheated at 40° C. for 10 min, and after preheating was completed, 200 U/g of partial glyceride lipase PCL was added. The obtained esterification product contained 13% of the fatty acids, 53% of the DAG, and 11.5% of the TAG. After separation and purification through molecular distillation, the content of the DAG was 81.20%.

### Example 6

(1) Hydrolysis: soybean oil and a phosphate buffer solution with a pH of 7.5 were added in a conical flask with a stopper, in a mass ratio of 1:0.5, were placed on a constant temperature magnetic stirrer at a speed of 500 rpm and preheated at 40° C. for 10 min, after preheating was completed, 500 U/g of TAG lipase MAS1-H108A (based on a total mass of oil) was added, after 6 h, sampling was performed for detection, standing for layering was performed, an oil phase was taken as a hydrolyzate, an aqueous phase was recovered, and the obtained oil phase did not need separation of fatty acids and glycerides. The obtained hydrolyzate contained 50.3% of the fatty acids, 25.5% of the DAG, and 18.2% of the TAG.

(2) Esterification: the hydrolyzate and the glycerol were added to the conical flask with a stopper in a mass ratio of (1:1), were placed on the constant temperature magnetic stirrer at a speed of 500 rpm and preheated at 40° C. for 10 min, and after preheating was completed, 200 U/g of partial glyceride lipase PCL was added. The obtained esterification product contained 7.6% of the fatty acids, 52.6% of the DAG, and 20.5% of the TAG. After separation and purification through molecular distillation, the content of the DAG was 72.90%.

### Example 7

(1) Hydrolysis: soybean oil and a phosphate buffer solution with a pH of 7.5 were added in a conical flask with a stopper, in a mass ratio of 1:0.5, were placed on a constant temperature magnetic stirrer at a speed of 500 rpm and preheated at 40° C. for 10 min, after preheating was completed, 500 U/g of TAG lipase MAS1 (based on a total mass of oil) was added, after 24 h, sampling was performed for detection, standing for layering was performed, an oil phase was taken as a hydrolyzate, an aqueous phase was recovered, and the obtained oil phase did not need separation of fatty acids and glycerides. The obtained hydrolyzate contained 48.3% of the fatty acids, 24.3% of the DAG, and 19.5% of the TAG.

(2) Esterification: the hydrolyzate and the glycerol were added to the conical flask with a stopper in a mass ratio of (1:1), were placed on the constant temperature magnetic stirrer at a speed of 500 rpm and preheated at 40° C. for 10 min, and after preheating was completed, 200 U/g of partial glyceride lipase PCL was added. The obtained esterification product contained 8.3% of the fatty acids, 50.20% of the DAG, and 21.5% of the TAG. After separation and purification through molecular distillation, the content of the DAG was 70.01%.

### Comparative Example 1

(1) Hydrolysis: soybean oil and a phosphate buffer solution with a pH of 7.5 were added in a conical flask with a stopper, in a mass ratio of 1:0.5, were placed on a constant temperature magnetic stirrer at a speed of 500 rpm and preheated at 40° C. for 10 min, after preheating was completed, 500 U/g of TAG lipase TL100L (based on a total mass of oil) was added, after 12 h, a hydrolyzate was sampled for detection, standing for layering was performed, an oil phase was taken as the hydrolyzate, an aqueous phase was recovered, and the obtained oil phase did not need separation of fatty acids and glycerides. The obtained hydrolyzate contained 65.8% of the fatty acids, 12.70% of the DAG, and 21.50% of the TAG.

(2) Esterification: the hydrolyzate and the glycerol were added to the conical flask with a stopper in a mass ratio of (1:1), were placed on the constant temperature magnetic stirrer at a speed of 500 rpm and preheated at 40° C. for 10 min, and after preheating was completed, 200 U/g of partial glyceride lipase PCL was added. The obtained esterification product contained 11.3% of the fatty acids, 45.2% of the DAG, and 21.4% of the TAG. After separation and purification through molecular distillation, the content of the DAG was 67.87%.

### Comparative Example 2

(1) Hydrolysis: soybean oil and a phosphate buffer solution with a pH of 7.5 were added in a conical flask with a stopper, in a mass ratio of 1:0.5, were placed on a constant temperature magnetic stirrer at a speed of 500 rpm and preheated at 40° C. for 10 min, after preheating was completed, 500 U/g of TAG lipase TL100L (based on a total mass of oil) was added, after 24 h, sampling was performed for detection, standing for layering was performed, an oil phase was taken as a hydrolyzate, an aqueous phase was recovered, and the obtained oil phase did not need separation of fatty acids and glycerides. The obtained hydrolyzate contained 70.2% of the fatty acids, 9.1% of the DAG, and 18.9% of the TAG.

(2) Esterification: the hydrolyzate and the glycerol were added to the conical flask with a stopper in a mass ratio of (1:1), were placed on the constant temperature magnetic stirrer at a speed of 500 rpm and preheated at 40° C. for 10 min, and after preheating was completed, 200 U/g of partial glyceride lipase PCL was added. The obtained esterification product contained 11.0% of the fatty acids, 44.8% of the DAG, and 19.2% of the TAG. After separation and purification through molecular distillation, the content of the DAG was 70.61%.

### Comparative Example 3

(1) Hydrolysis: soybean oil and a phosphate buffer solution with a pH of 7.5 were added in a conical flask with a stopper, in a mass ratio of 1:0.5, were placed on a constant temperature magnetic stirrer at a speed of 500 rpm and preheated at 40° C. for 10 min, after preheating was completed, 500 U/g of TAG lipase TL100L (based on a total mass of oil) was added, after 36 h, sampling was performed for detection, standing for layering was performed, an oil phase was taken as a hydrolyzate, an aqueous phase was recovered, and the obtained oil phase did not need separation of fatty acids and glycerides. The obtained hydrolyzate contained 79.1% of the fatty acids, 6.0% of the DAG, and 14.9% of the TAG.

(2) Esterification: the hydrolyzate and the glycerol were added to the conical flask with a stopper in a mass ratio of (1:1), were placed on the constant temperature magnetic stirrer at a speed of 500 rpm and preheated at 40° C. for 10 min, and after preheating was completed, 200 U/g of partial glyceride lipase PCL was added. The obtained esterification product contained 14.1% of the fatty acids, 43.5% of the DAG, and 15.9% of the TAG. After separation and purification through molecular distillation, the content of the DAG was 72.50%.

### Comparative Example 4

(1) Hydrolysis: soybean oil and a phosphate buffer solution with a pH of 7.5 were added in a conical flask with a stopper, in a mass ratio of 1:0.5, were placed on a constant temperature magnetic stirrer at a speed of 500 rpm and preheated at 40° C. for 10 min, after preheating was completed, 500 U/g of TAG lipase TL100L (based on a total mass of oil) was added, after 8 h, sampling was performed for detection, standing for layering was performed, an oil phase was taken as a hydrolyzate, an aqueous phase was recovered, and the obtained oil phase did not need separation of fatty acids and glycerides. The obtained hydrolyzate contained 49.5% of the fatty acids, 21.9% of the DAG, and 28.01% of the TAG.

(2) Esterification: the hydrolyzate and the glycerol were added to the conical flask with a stopper in a mass ratio of (1:1), were placed on the constant temperature magnetic stirrer at a speed of 500 rpm and preheated at 40° C. for 10 min, and after preheating was completed, 200 U/g of partial glyceride lipase PCL was added. The obtained esterification product contained 8.2% of the fatty acids, 45.8% of the DAG, and 29.2% of the TAG. After separation and purification through molecular distillation, the content of the DAG was 61.30%.

TABLE 1
Content of substances at different stages in examples and comparative examples

| | | Hydrolysis | | | | | | | Molecular |
|---|---|---|---|---|---|---|---|---|---|
| | Lipase in a | process | | | Esterification process | | | | distillation |
| | hydrolysis | TAG | FFA | DAG | TAG | DAG | MAG | FFA | DAG |
| Case | process | (%) | (%) | (%) | (%) | (%) | (%) | (%) | (%) |
| Example 1 | MAS1-H108A | 12.3 | 60.2 | 20.5 | 12.1 | 55.4 | 22.9 | 9.5 | 82.07 |
| Example 2 | MAS1-H108A | 10.5 | 70.1 | 15.6 | 10.0 | 53.0 | 23.8 | 13.2 | 84.13 |
| Example 3 | MAS1-H108A | 8.2 | 80.8 | 10.1 | 8.1 | 50.2 | 26.5 | 15.1 | 85.79 |
| Example 4 | MAS1 | 13.3 | 63.0 | 18.7 | 13.1 | 52.3 | 22.4 | 12.20 | 80.02 |
| Example 5 | MAS1 | 11.8 | 73.0 | 13.1 | 11.5 | 53.0 | 22.5 | 13.00 | 81.20 |
| Example 6 | MAS1-H108A | 18.2 | 50.3 | 25.5 | 20.5 | 52.6 | 19.3 | 7.6 | 72.90 |
| Example 7 | MAS1 | 19.5 | 48.3 | 24.3 | 21.5 | 50.20 | 20.0 | 8.3 | 70.01 |
| Comparative | TL100L | 21.5 | 65.8 | 12.7 | 21.4 | 45.2 | 22.1 | 11.3 | 67.87 |
| Example 1 | | | | | | | | | |
| Comparative | TL100L | 18.9 | 70.2 | 9.1 | 19.2 | 44.8 | 25.0 | 11.0 | 70.61 |
| Example 2 | | | | | | | | | |
| Comparative | TL100L | 14.9 | 79.1 | 6.0 | 15.9 | 43.5 | 26.1 | 14.1 | 72.50 |
| Example 3 | | | | | | | | | |
| Comparative | TL100L | 28.01 | 49.5 | 21.9 | 29.2 | 45.8 | 16.8 | 8.20 | 61.30 |
| Example 4 | | | | | | | | | |

From results in Table 1, it can be seen that the method for synthesizing DAG in Examples 1-5 of the present disclosure adopts a method of first hydrolysis and then esterification, besides, in a hydrolysis process, the TAG lipase MAS1 or MAS1-H108A is used for catalytic hydrolysis, and the content of the fatty acids in the hydrolyzate is controlled to be greater than 60%. Then, the obtained hydrolyzate is subjected to esterification, and the content of the DAG in the esterification product is greater than 50%. The esterification product is purified by simple molecular distillation, and the content of the DAG is greater than 80%. Herein, the content of the DAG in products obtained by a method of using a mutant of lipase MAS1-H108A in Examples 1-3 is significantly higher than that by a method of using the wild-type lipase MAS1 in Examples 4-5.

In Example 6, the content of the fatty acids in the hydrolyzate is controlled to be 50.3%, and the content of the obtained DAG is reduced compared with that in Examples 1-3. In Example 7, the content of the fatty acids in the hydrolyzate is controlled to be 48.3%, and the content of the DAG is reduced compared with that in Examples 4-5. It is indicated that in the method disclosed by the present disclosure, the content of the fatty acids in the hydrolyzate is controlled to be greater than 60%, and higher-purity DAG can be obtained.

In the Comparative Examples 1-4, the TAG lipase TL100L is selected for catalytic hydrolysis. In the Comparative Examples 1-3, even if the content of the fatty acids in the hydrolyzate is controlled to be greater than 60%, the purity of the obtained DAG is much lower than that in Examples 1-5. In Comparative Example 4, the content of the fatty acids in the hydrolyzate is controlled to be 49.5%, and the purity, namely 61.30%, of the DAG is lower than that in all examples.

From the results in Table 1, it can also be seen that in Examples 1-5, in the hydrolysis course, when the TAG lipase MAS1 or MAS1-H108A is selected for catalytic hydrolysis, the content of TAG in the hydrolyzate is significantly lower than that of DAG. However, in the hydrolysis process of Comparative Examples 1-4, when the TAG lipase TL100L is selected for catalytic hydrolysis, the content of the TAG in the hydrolyzate is significantly higher than that of the DAG under the same content of the fatty acids, and the content of the TAG affects the separation and the purification, ultimately affecting the purity of the DAG.

Based on this, the present disclosure performs further in-depth research on the hydrolysis of the oil with the TAG lipase MAS1, MAS1-H108A, and TL100L. The contents of various substances at different time points after reaction for 1-108 h are measured, and the results are shown in Tables 2-4.

TABLE 2
Contents of various substances after enzymatic hydrolysis
with MAS1-H108A at different reaction time points

| | TAG | FFA | 1,3-DAG | 1,2-DAG | DAG | 1-MAG | 2-MAG |
|---|---|---|---|---|---|---|---|
| Time/h | % | % | % | % | % | % | % |
| 1 | 74.58 | 10.20 | 8.78 | 6.43 | 15.21 | — | — |
| 2 | 55.90 | 20.56 | 12.25 | 9.65 | 21.90 | 1.64 | |
| 4 | 38.22 | 31.98 | 13.90 | 10.56 | 24.46 | 4.23 | 1.10 |
| 6 | 29.44 | 38.41 | 13.97 | 10.85 | 24.82 | 6.02 | 1.30 |
| 8 | 25.01 | 42.86 | 13.64 | 11.13 | 24.77 | 5.96 | 1.40 |
| 10 | 20.85 | 46.21 | 13.61 | 11.03 | 24.64 | 7.02 | — |
| 12 | 18.83 | 49.30 | 13.03 | 10.66 | 23.69 | 6.94 | 1.24 |
| 24 | 12.85 | 59.95 | 11.45 | 8.54 | 19.99 | 6.52 | 0.69 |
| 48 | 10.17 | 69.90 | 8.98 | 5.84 | 14.82 | 4.57 | 0.54 |
| 72 | 8.85 | 80.42 | 5.84 | 3.35 | 9.19 | 1.55 | |

Reaction conditions: water: soybean oil-1:2 (in a mass ratio), buffer with a pH of 7, temperature being 40° C., and addition quantity of TAG lipase MAS1-H108A being 200 U/g.

TABLE 3
Contents of various substances after enzymatic hydrolysis
with MAS1 at different reaction time points

| | TAG | FFA | 1,3-DAG | 1,2-DAG | DAG | 1-MAG | 2-MAG |
|---|---|---|---|---|---|---|---|
| Time/h | % | % | % | % | % | % | % |
| 1 | 84.5 | 6.05 | 5.6 | 3.8 | 9.4 | 0.05 | |
| 2 | 75.2 | 10.8 | 7.5 | 5.9 | 13.4 | 0.6 | |
| 4 | 65.2 | 16.5 | 9.1 | 7.8 | 16.9 | 1.28 | 0.12 |
| 6 | 49.4 | 26.7 | 12.5 | 8.3 | 20.8 | 2.9 | 0.2 |
| 8 | 38.5 | 30.5 | 13.1 | 11.4 | 24.5 | 5 | 1.5 |
| 10 | 30.4 | 37.2 | 12.5 | 10.8 | 23.3 | 7.8 | 1.3 |
| 12 | 26.8 | 41.9 | 12.7 | 10.5 | 23.2 | 6.5 | 1.6 |
| 24 | 21.5 | 45.9 | 12.6 | 10.6 | 23.2 | 7.6 | 1.8 |
| 48 | 19.5 | 49.8 | 12.5 | 8.8 | 21.3 | 7.8 | 1.6 |
| 72 | 14.3 | 59.5 | 10.5 | 7.9 | 18.4 | 6.3 | 1.5 |

Reaction conditions: water: soybean oil=1:2 (in a mass ratio), buffer with a pH of 7, temperature being 40° C., and addition quantity of TAG lipase MAS1 being 200 U/g.

TABLE 4
Contents of various substances after enzymatic hydrolysis
with TL100L at different reaction time points

| | TAG | FFA | 1,3-DAG | 1,2-DAG | DAG | 2-MAG |
|---|---|---|---|---|---|---|
| Time/h | % | % | % | % | % | % |
| 1 | 27.25 | 33.58 | 0.39 | 30.86 | 31.25 | 7.92 |
| 2 | 27.54 | 35.19 | 0.48 | 31.01 | 31.49 | 5.78 |
| 4 | 27.91 | 36.34 | 0.79 | 29.82 | 30.61 | 5.14 |
| 6 | 28.96 | 38.72 | 0.91 | 28.70 | 29.61 | 2.71 |
| 8 | 29.41 | 41.08 | 1.25 | 25.33 | 26.58 | 2.93 |
| 10 | 29.36 | 44.58 | 0.99 | 22.91 | 23.90 | 2.16 |
| 12 | 27.91 | 48.09 | 0.60 | 21.02 | 21.62 | 2.38 |
| 24 | 21.54 | 65.80 | 0.87 | 11.79 | 12.66 | — |
| 36 | 15.96 | 75.00 | 1.26 | 7.79 | 9.05 | — |
| 48 | 14.47 | 80.28 | 0.45 | 4.57 | 5.02 | |

Reaction conditions: water: soybean oil=1:2 (in a mass ratio), buffer with a pH of 7, temperature being 40° C., and addition quantity of TAG lipase TL 100 being 200 U/g.

The results of hydrolyzing the soybean oil with the MAS1-H108A, the MAS1, and the TL100L are compared, and it is found that the MAS1-H108A and the MAS1 more tend to hydrolyze TAG in the hydrolysis process. When the content of the fatty acids reaches 45% or above, the content of the DAG in the hydrolyzate is greater than that of the TAG. When hydrolysis continues, it is found that when the content of the fatty acids is greater than 60%, the content of the TAG will continue to decrease. The obtained hydrolyzate can be directly subjected to the esterification reaction with the glycerol under the catalysis of the partial glyceride lipase without separating the TAG from the fatty acids, and higher-purity DAG can be prepared. When the soybean oil is hydrolyzed with the TL100L, the content of the TAG is almost always higher than that of the DAG, and the content of the TAG in the obtained hydrolyzate is also significantly higher than that of the DAG.

Various technical features of the above examples can be combined freely. To make the description concise, all possible combinations of the various technical features in the above examples have not been described. However, as long as there is no contradiction in the combination of these technical features, they should be considered within the scope of this description.

The above-mentioned examples only express several implementations of the present disclosure, which are described more specifically in detail, but they cannot be construed as limitations to the scope of the present patent. It should be noted that for those of ordinary skill in the art, without deviating from the concept of the present disclosure, a number of variations and improvements can be made, which are within the scope of protection of the present disclosure. Therefore, the protection scope of the present patent should be defined by the appended claims.

1. A method for synthesizing diacylglycerol (DAG), comprising the following steps:
step (1), mixing an oil, a lipase and a solvent, and performing a hydrolysis reaction to obtain a hydrolyzate; and
step (2), under the action of a partial glyceride lipase, performing an esterification reaction on the hydrolyzate obtained in step (1) with glycerol, wherein
the lipase in step (1) is lipase MAS1 or MAS1-H108A.

2. The method for synthesizing DAG according to claim 1, wherein content of fatty acids in the hydrolyzate obtained in step (1) is controlled to be greater than 45 wt %, preferably greater than 60 wt %.

3. The method for synthesizing DAG according to claim 1, wherein content of triacylglycerol (TAG) in the hydrolyzate obtained in step (1) is controlled to be less than 22 wt %, preferably less than 20 wt %.

4. The method for synthesizing DAG according to claim 1, wherein the partial glyceride lipase is one or a mixture of more than two of lipase SMG1, lipase PCL and lipase AOL.

5. The method for synthesizing DAG according to claim 1, wherein the solvent is water or a buffer with a pH of 6-8.

6. The method for synthesizing DAG according to claim 1, wherein the temperature of the hydrolysis reaction in step (1) is 20-60° C., and the pH of the hydrolysis reaction is 6-8; and/or, the temperature of the esterification reaction in step (2) is 30-50° C., and the pH of the esterification reaction is 6-8.

7. The method for synthesizing DAG according to claim 1, wherein a mass ratio of the oil to the solvent in step (1) is 1:(0.1-5), and further preferably the mass ratio is 1:(0.1-2);
and/or, the mass ratio of the glycerol to the hydrolyzate in step (2) is (0.5-5):1, further preferably (0.5-1.5):1.

8. The method for synthesizing DAG according to claim 1, wherein the hydrolyzate is an oil phase obtained by layering a reaction system after the hydrolysis reaction is completed.

9. The method for synthesizing DAG according to claim 1, wherein after the obtained esterification reaction in step (2) is completed, an esterification product is subjected to separation and purification, and further, the separation and the purification are separation and purification through molecular distillation.

10. Diacylglycerol (DAG) prepared by the method for synthesizing DAG according to claim 1.
